(19) United States
(12) Patent Application Publication
(10) Pub. No.: US 2022/0023158 A1
(43) Pub. Date: Jan. 27, 2022

(54) TEMPORARY TATTOO DECALS USABLE AS DOSIMETERS

(71) Applicants: Andrew Seth LEVINE, Lutherville, MD (US); Nicole Ann ZUJOVIC, Baltimore, MD (US); Debora HENSE, Plymouth, MI (US)

(72) Inventors: Andrew Seth LEVINE, Lutherville, MD (US); Nicole Ann ZUJOVIC, Baltimore, MD (US); Debora HENSE, Plymouth, MI (US)

(21) Appl. No.: 17/300,115

(22) PCT Filed: Feb. 22, 2019
(86) PCT No.: PCT/US2019/019204

§ 371 Date: Mar. 15, 2021

Related U.S. Application Data
(60) Provisional application No. 62/633,632, filed on Feb. 22, 2018.

(51) Int. Cl. A61K 8/02; A61Q 1/02

(52) U.S. Cl. CPC A61K 8/0204; A61Q 1/02

(57) ABSTRACT

A temporary tattoo decal including: (a) a porous paper base; (b) a water soluble slip layer or a release coating on at least a first surface of the paper base; (c) a photochromic ink layer atop the slip layer or the release coating; and (d) a pressure-sensitive adhesive layer atop the photochromic ink layer, wherein the photochromic ink layer includes at least one photochromic material that is capable of changing its color when exposed to UV radiation and a polymer matrix in which the at least one photochromic material is distributed; and wherein the polymer matrix substantially prevents the ink material from degrading when covered with a sunscreen product and inhibits the photochromic ink from washing off when the sunscreen product covering the photochromic ink layer is washed off.

### FIELD OF THE INVENTION

The present invention pertains generally to temporary tattoo decals that comprise photochromic material so that the decals can be used as dosimeters and to methods for their use.

### BACKGROUND OF THE INVENTION

Decals have been used for many decades for decorative effect on a variety of objects. Decals have also been used as temporary tattoos for application to skin. For example, U.S. Pat. No. 4,522,864 issued to Humason teaches a decal having a porous paper backing, a water-soluble slip layer on the surface of the paper backing, a microscopically thin, flexible, extensible, water-resistant film covering the slip layer, a very thin translucent multi-color design offset (lithographically) printed on the water-resistant film, and a uniform layer of transparent or translucent pressure sensitive adhesive covering the design.

As another example, U.S. Pat. No. 6,074,721 to Moore teaches a temporary decal comprising a porous paper base coated with a water-soluble slip layer, e.g., dextrin gum, on which offset printing inks are directly imprinted using a high speed lithographic process. The inks are printed in a designated area only, in a desired image. A coating and a contact adhesive is then printed over the image area.

Decals have also been described which comprise photochromic materials so that they can be used as dosimeters. For example, U.S. Pat. No. 8,961,897 to Faran describes a dosimeter having a top layer comprising a photochromic ink composition in a polymer matrix, an opaque layer under the top layer and a bottom layer made of a sticky material for attaching to a user's skin. U.S. Pat. No. 8,501,053 to Jeganathan describes a photoactive color changing ink, coating, plastic or printed article comprising a photochromic dye, wherein in one embodiment the article is in the form of a temporary tattoo. U.S. Pat. No. 6,531,118 to Gonzalez describes a topical composition having a reversible visible change in color in response to a change in light comprising a photochromic material and a vehicle acceptable for topical application to the skin of a user. In one embodiment, the composition can be a tattoo product. US 2002/0022008 to Forest describes a sun protection kit and indicator comprising (a) a sunscreen and (b) an indicator comprising a photochromic molecule and a carrier, wherein the indicator comprises a solid film that is a temporary tattoo.

The above patents and patent application publications (which are hereby incorporated herein by reference) do not consider that the components typically found in sunscreens, including titanium dioxide and zinc oxide, may be photoactive, particularly when present as nanoparticles. They have the ability to absorb UV radiation, and in the presence of moisture convert water molecules into chemically active hydroxyl free radicals. These highly reactive chemicals are highly corrosive and, if sunscreen is used with a tattoo comprising a layer of photochromic material and comes in contact with a surface of a polymer matrix comprising the photochromic material, the sunscreen could etch the surface of the photochromic layer resulting in the sunscreen becoming a permanent part of the matrix material. This could prevent UV radiation from coming in contact with the photochromic material and thereby prevent a color change from occurring.

Accordingly, what is needed is a tattoo decal for use with sunscreen that either has a matrix/photochromic layer that is capable of withstanding the corrosive effects of sunscreen or that has a layer of material that is resistant to sunscreen or that is capable of absorbing a minimal amount of sunscreen but that nevertheless protects a matrix/photochromic layer of a tattoo decal from contact with the sunscreen so absorbed.

### SUMMARY OF THE INVENTION

In a first embodiment of the invention, there is provided a temporary tattoo decal comprising:
(a) a porous paper base;
(b) a water soluble slip layer or a release coating on at least a first surface of the paper base;
(c) a photochromic ink layer atop the slip layer or the release coating; and
(d) a pressure-sensitive adhesive layer atop the photochromic ink layer;

wherein the photochromic ink layer comprises at least one photochromic material that is capable of changing its color when exposed to UV radiation and a polymer matrix in which the at least one photochromic material is distributed; and wherein the polymer matrix substantially prevents the ink material from degrading when covered with a sunscreen product and inhibits the photochromic ink from washing off when the sunscreen product covering the photochromic ink layer is washed off; and wherein the temporary tattoo decal is constructed and arranged such that, when the porous paper base is wetted with water with the pressure-sensitive adhesive layer of the temporary tattoo decal pressed against the skin of a user, the porous paper base and slip layer or release coating can be separated from a remainder of the temporary tattoo decal leaving a photochromic ink image adhered to the skin of the user via the pressure-sensitive adhesive layer. In a preferred embodiment, the polymer matrix comprises a polymer selected from the group consisting of shellac, polyurethane, acrylic polymer, silicone or a mixture thereof. In a most preferred embodiment, the polymer matrix comprises shellac.

The temporary tattoo decal can further comprise a barrier layer between the photochromic ink layer and the pressure-sensitive adhesive layer that prevents the photochromic material in the photochromic ink layer from possibly migrating to the pressure-sensitive adhesive layer. In a preferred embodiment, the barrier layer comprises shellac, acrylic, acrylate, polyurethane or cellulose.

The temporary tattoo decal can further comprise a removable release liner on the pressure-sensitive adhesive layer and a transparent sunscreen resistant layer between the slip layer or release coating and the photochromic ink layer. The transparent sunscreen resistant layer can preferably comprise a polymer selected from the group consisting of shellac, acrylic, acrylate, polyurethane, silicone and siloxane.

In a second embodiment of the invention, there is provided a temporary tattoo decal comprising:
(a) a porous paper base;
(b) a slip layer or release coating on at least a first surface of the paper base;
(c) a transparent clear coat layer atop the slip layer or release coating;
(d) a photochromic ink layer atop the transparent clear coat layer, wherein the photochromic ink layer comprises (i) at least one photochromic material that is capable of changing its color when exposed to UV radiation and (ii) a polymer matrix in which the at least one photochromic material is distributed; and
(e) a pressure-sensitive adhesive layer covering the photochromic ink layer,

wherein the temporary tattoo decal is constructed and arranged such that, when the porous paper base is wetted with water with the temporary tattoo decal disposed with the pressure-sensitive adhesive layer pressed against the skin of a user, the porous paper base and slip layer or release coating can be separated from a remainder of the temporary tattoo decal leaving a photochromic ink image adhered to the skin of the user via the pressure-sensitive adhesive layer, and wherein the transparent clear coat layer comprises a material that is constructed and arranged to allow at least a portion of a sunscreen product, when deposited on a top surface of the clear coat layer, to adhere just well enough to a surface of the clear coat layer to filter UV light from the photochromic layer below, the clear coat layer being transparent to allow UV radiation to pass through the clear coat layer to the photochromic ink layer at least when the surface of the clear coat layer is not covered with the sunscreen product. In a preferred embodiment, the polymer matrix comprises a polymer selected from the group consisting of shellac, polyurethane, acrylic polymer and a mixture thereof.

The temporary tattoo decal according to the second embodiment can further comprise a barrier layer between the photochromic ink layer and the pressure-sensitive adhesive layer that prevents the photochromic material in the photochromic ink layer from possibly migrating to the pressure-sensitive adhesive layer. The barrier layer can comprise, for example, shellac, acrylic, acrylate, polyurethane or cellulose.

The temporary tattoo decal according to the second embodiment can further comprise a removable release liner on the pressure-sensitive adhesive layer. The removable release liner can comprise, for example, a silicone-coated polyester film.

The temporary tattoo decal according to the second embodiment can comprise a clear coat layer comprising a polymer selected from the group consisting of shellac, acrylic, acrylate, polyurethane, silicone and siloxane.

In accordance with a third embodiment of the invention, there is provided a temporary tattoo decal comprising:
(a) a carrier film with pressure-sensitive adhesive coating on a first surface thereof;
(b) a photochromic ink layer on a surface of the carrier film that is opposed to the first surface comprising the pressure-sensitive adhesive coating; wherein the photochromic ink layer comprises at least one photochromic material that is capable of changing its color when exposed to UV radiation and a polymer matrix in which the at least one photochromic material is distributed; and wherein the polymer matrix substantially prevents the ink material from degrading when covered with a sunscreen product and inhibits the photochromic ink from washing off when the sunscreen product covering the photochromic ink layer is washed off.

The temporary tattoo decal according to the third embodiment can preferably comprise a release liner removably attached to the pressure sensitive adhesive coating to protect the pressure sensitive adhesive coating. The release liner can preferably comprise a silicone-coated polyester film or paper.

The temporary tattoo decal according to the third embodiment can comprise a transparent clear coat layer atop the photochromic ink layer to prevent sunscreen applied to the temporary tattoo decal from penetrating to the photochromic ink layer. The clear coat layer preferably comprises a polymer selected from the group consisting of shellac, acrylic, acrylate, polyurethane, silicone and siloxane.

### BRIEF DESCRIPTION OF THE DRAWINGS

FIG. 1 is a depiction of tattoo constructions 1A-C, 2A-C and 3A-C representing different possible arrangements of the layers in preferred tattoo construction types of the invention; and

FIG. 2 is a depiction showing preferred materials and thicknesses of the layers in preferred tattoo construction types.

### DETAILED DESCRIPTION

The temporary tattoo decals of the invention can be divided into two (2) basic construction types depending on how the decals are attachable to the skin of a user.

First Construction Type

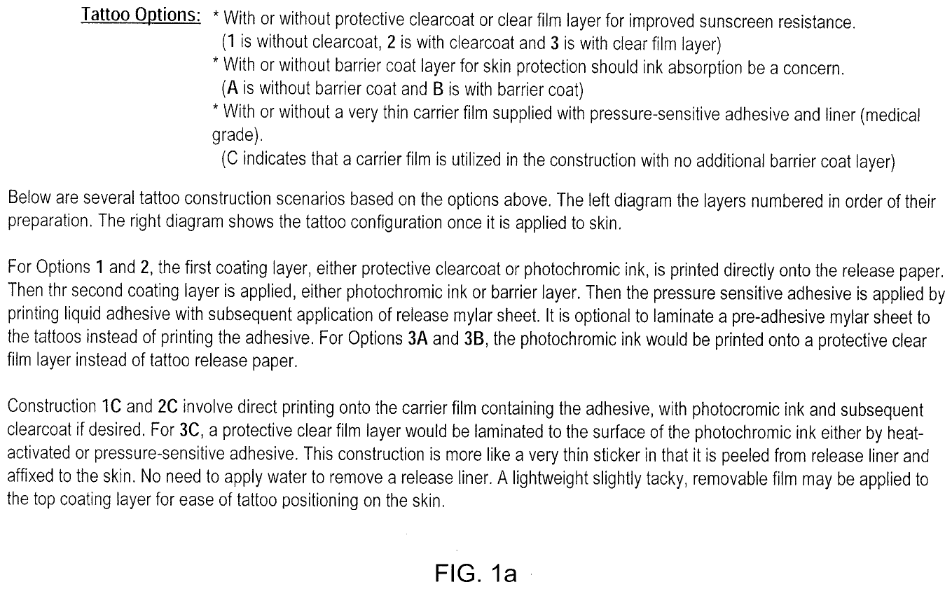

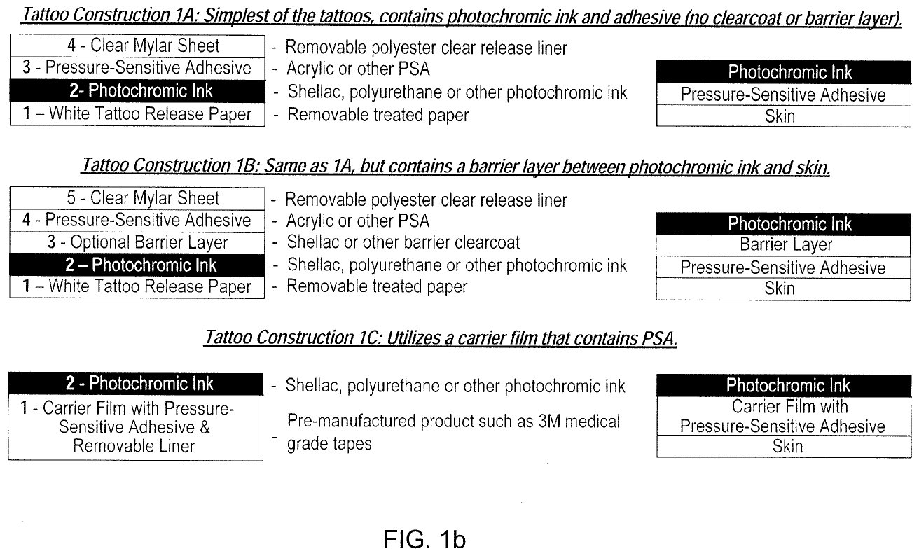

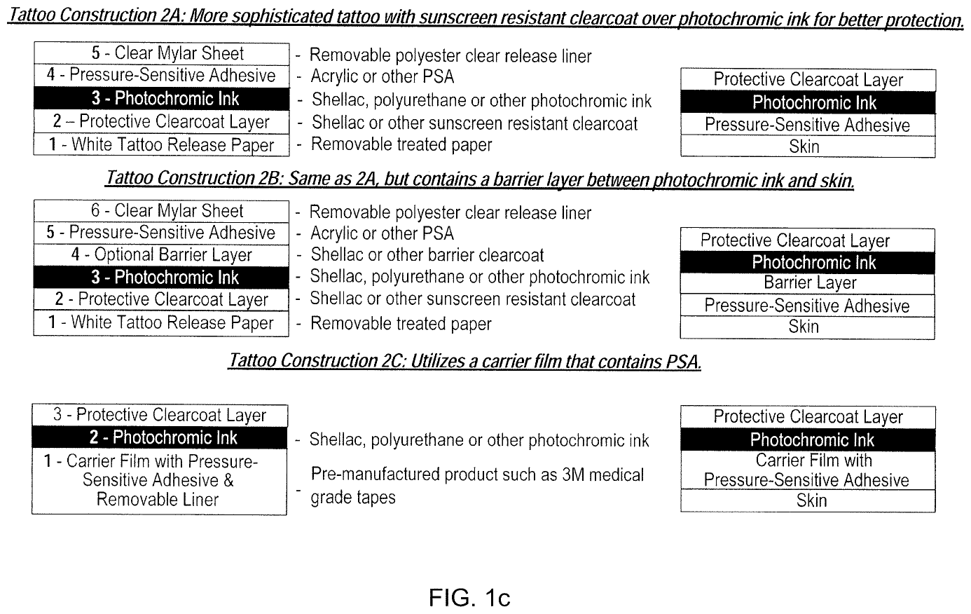

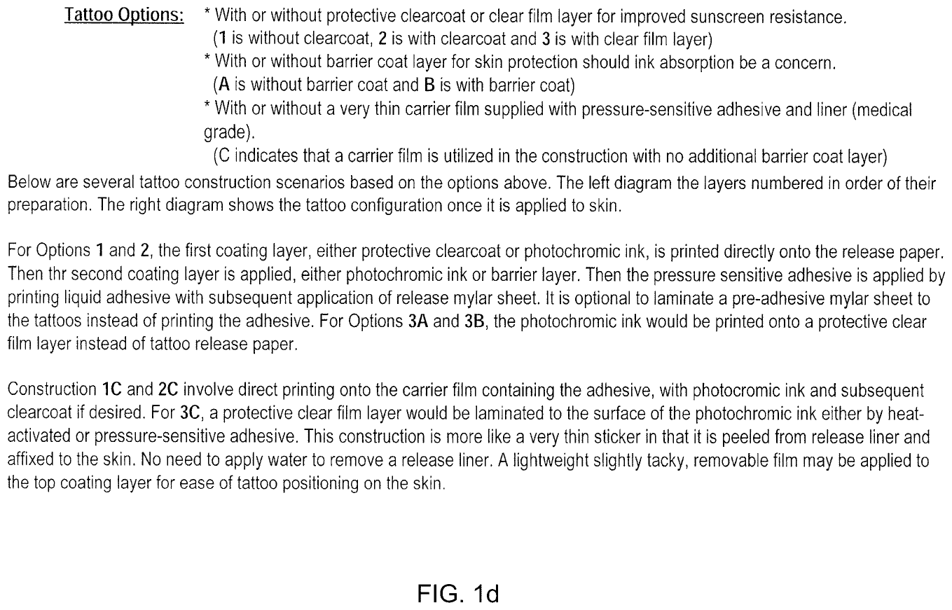

In a first construction type, the decals use a porous paper base comprising (usually, just one side) a release coating, such as non-stick silicone, which facilitates separation of the paper base from a photochromic ink layer printed thereon. A transfer film comprising, for example, gelatin or other polymeric materials can be printed or otherwise formed on the non-stick silicone. Alternatively, the paper base can be coated with a water-soluble slip layer, which may be, for example, dextrin gum, which dissolves when the paper base is saturated with water to effect separation of the paper base from the photochromic ink layer. The combination of the porous paper base with non-stick release layer or with a slip layer is available as standard prefabricated decal paper from a variety of sources. A preferred tattoo paper is commercially available from One Step Papers, LC under the trademark TRANSLIDE®“T” ECO temporary Tattoo Transfer Paper. A photochromic ink layer printed on the prefabricated decal paper can be separated therefrom by wetting the prefabricated decal paper with water. Preferred embodiments of this first construction type are described next with reference to tattoo constructions 1A, 1B, 2A and 2B in FIG. 1 attached.

In each of these embodiments, the left side diagram shows the layers sequenced in order of their preparation wherein the right side diagram shows the layers sequenced in the configuration in which they are applied to the skin of a user.

The embodiment of construction 1A depicts a basic form of the first type of construction wherein photochromic ink is printed directly atop the decal paper with pressure sensitive adhesive being coated atop the photochromic ink. A releasable cover sheet or liner comprising, for example, a polyester such as Mylar can be layered atop the pressure-sensitive adhesive to protect it until the tattoo decal is ready for application whereupon it can be taken off, as shown on the right side diagram. The pressure-sensitive adhesive can be an acrylic or any other pressure sensitive adhesive. The photochromic ink can comprise a photochromic pigment or dye in a polymer matrix, such as shellac or polyurethane as discussed further below.

The embodiment of construction 1B is the same as that of construction 1A, except a barrier layer is added between the photochromic ink layer and the pressure-sensitive adhesive layer (left side diagram). This additional layer prevents the photochromic ink from penetrating into and through the pressure-sensitive adhesive layer and onto the skin of a user (right side diagram). The barrier layer may be made, for example, of shellac, acrylic or another barrier material.

The embodiment of construction 2A is the same as that of construction 1A, except a transparent clear coat layer is added between the photochromic ink layer and the decal paper layer (left side diagram). The clear coat layer is adapted to protect the photochromic ink layer from degradation from sunscreen that may be applied atop the tattoo after it is applied to the skin of a user (right side diagram).

The embodiment of construction 2B is the same as that of construction 2A, except a transparent clear coat layer is added between the photochromic layer and the decal paper layer (left side diagram). The clear coat layer is adapted to protect the photochromic ink layer from degradation from sunscreen that may be applied atop the tattoo after it is applied to the skin of a user (right side diagram).

To apply a temporary tattoo decal of this type of construction to the skin of a user, the clear Mylar cover sheet would be removed and the contact adhesive side of the decal would be placed on the skin. The tattoo release paper comprising the porous paper base would then be pressed against the skin and wetted or saturated with water to facilitate removal of the release paper or dissolution of the slip layer and to cause transfer of a photochromic image to the skin. A preferred method of applying the tattoo to the skin of a user comprises the following steps:
1. preferably the skin would be completely clean of oils and makeup;
2. any Mylar or other cover sheet would be removed from the tattoo decal and discarded;
3. the tattoo decal would be placed face down on the clean and, preferably, very dry skin;
4. the back of the tattoo decal would be wetted with a very wet cloth or water, and firm pressure would be applied to the back of the tattoo decal;
5. after a waiting period, preferably lasting about 30 seconds, the paper backing would be slid off; and
6. optionally, the tattoo would be washed gently with soap and water for a more realistic look.

Second Construction Type

In the second construction type, the photochromic ink is printed either (a) on a thin carrier film supplied with pressure sensitive adhesive and, optionally, removable liner (medical grade) in a single, all-in-one product, or (b) on a protective clear film that, optionally, has a pressure sensitive adhesive and liner. Pre-manufactured products of the thin carrier film are available, for example, from the Minnesota Mining and Manufacturing Company (3M) of Maplewood, Minn. With this construction type, the photochromic ink layer is printed or otherwise applied directly onto the carrier film with adhesive or onto the protective clear film layer. This construction is like a very thin sticker in that the tattoo decal is peeled from a release liner and affixed to the skin of a user. There is no need to apply water to remove the release liner. A lightweight slightly tacky, removable film may be applied to the top coating layer for case of tattoo positioning on the skin of a user. Preferred embodiments of this type of construction are described next with reference to FIG. 1.

The embodiment of construction 1C depicts a basic form of this type of construction wherein photochromic ink is printed directly onto the carrier film (left side diagram). The release liner can be peeled from the carrier film and the decal is pressed against the skin of the user with the layers in the same sequence (see right side diagram).

The embodiment of construction 2C is the same as that of construction 1C, except that a transparent clear coat layer is coated or otherwise deposited atop the photochromic ink layer (left side diagram). The clear coat layer is adapted to protect the photochromic ink layer from degradation from sunscreen that may be applied atop the tattoo after it is applied to the skin of a user (right side diagram).

In the embodiments of construction 3A, 3B and 3C, photochromic ink is applied atop a protective clear film layer with optional carrier film (left side diagrams). The protective clear film layer protects the photochromic ink layer from, e.g., degradation due to sunscreen and can be have the same composition as the clear coat layer described above.

For options 3A and 38, the photochromic ink may preferably be printed onto the protective clear film layer. For option 3C, the protective clear film layer may preferably be laminated to a surface of the photochromic ink by heat-activated or pressure-sensitive adhesive. In embodiment 3A, a pressure-sensitive adhesive layer is applied atop the photochromic ink followed by a release liner (left side diagram). In application, the release liner is peeled away and the pressure-sensitive adhesive layer is pressed against the skin of a user with the protective clear film layer atop and protecting the photochromic ink (right side diagram).

The embodiment of construction 3B is the same as that of construction 3A, except a barrier layer is added between the photochromic ink layer and the pressure-sensitive adhesive layer (left side diagram). This additional layer prevents the photochromic ink from penetrating into and through the pressure-sensitive adhesive layer and onto the skin of a user (right side diagram). The barrier layer may be made, for example, of shellac, acrylic or another barrier material.

Embodiment 3C differs from embodiment 3A in that it is provided with an all-in-one carrier film layer, including pressure sensitive adhesive and removable liner top the photochromic ink layer, instead of the pressure-sensitive adhesive layer and release liner as in embodiment 3A (left side diagram). In application, the removable liner is removed from the carrier film layer and the carrier film layer with pressure-sensitive adhesive is pressed against the skin of a user with the protective clear film layer atop the photochromic ink layer (right side diagram).

Photochromic Ink

The photochromic inks for use in the photochromic ink layers of the above construction types comprise (i) at least one photochromic material that is capable of changing its color when exposed to UV radiation; (ii) a polymer matrix in which the at least one photochromic material is distributed; and optionally (iii) a component or components that change a rate at which the at least one photochromic material changes its color when exposed to UV radiation or inhibits the at least one photochromic material from reverting to its original color. The matrix is preferably one that (a) substantially prevents the ink composition from degrading when covered with the sunscreen product and (b) inhibits the ink composition from washing off when the sunscreen is washed off. The matrix is preferably selected from the group consisting of shellac, polyurethane, acrylic polymer, silicone or a mixture thereof. The matrix most preferably comprises shellac.

The photochromic material can be either a photochromic pigment or a photochromic dye. In accordance with one preferred embodiment of the invention, the photochromic pigment or dye is selected such that the color change is reversible. In accordance with this embodiment, the at least one reversible photochromic pigment or dye that is either (a) colorless in an inactive state, activates to color when exposed to ultraviolet (UV) light, reverts to colorless when covered with the selected sunscreen product and re-activates to color when the sunscreen product is removed, or (b) an initial color in an inactive state, activates to colorless or to a different color when exposed to ultraviolet (UV) light, reverts to the initial color when covered with the selected sunscreen product and re-activates to colorless or to the different color when the sunscreen product is removed. In a preferred embodiment, the at least one pigment or dye can comprise a naphthoxazine-based photochromic dye that yields green ink when dried, but turns to a brilliant blue in the presence of sunlight.

In another preferred embodiment, the photochromic pigment or dye and the other components of the ink composition may be selected such that the color change of the at least one pigment or dye is irreversible in that the color of the photochromic pigment or dye would not reverse after it has been exposed to a predetermined amount of UV radiation. In this embodiment, an initial color of the photochromic pigment or dye can fade from a bright, activated color to a faded color after it has been exposed to a predetermined amount of UV radiation.

The photochromic material can comprise any of the photochromic pigments or dyes described in Applicant's PCT application, PCT/US17/47751, filed 21 Aug. 2017. For example, the photochromic material can comprise a pigment or dye selected from the group consisting of oxazines, naphtoxazines, spiro-naphthoxazines, pyrans, spiropyrans, napthopyrans, spironapthopyrans, fulgides, diarylethenes having heterocyclic aryl groups, dihydroindolizines, quinones, perimidinespirocycloheadienones, viologens and mixtures thereof.

The photochromic material and other components can be incorporated within the matrix by means of any known-in-the-art suitable method, for example by extruding, molding, casting or printing. Although the aforementioned matrixes are preferred in all of the construction types because they protect the photochromic layer from degradation by sunscreen applied onto the temporary tattoo decals, other matrixes can be advantageously used in the construction types that comprise a transparent clear coat layer that protects the photochromic layer from sunscreen, as in constructions 2A, 28 and 2C depicted above. The transparent clear coat layer can preferably comprise shellac, acrylic/acrylate, polyurethane, silicone, siloxane or low density polyethylene.

### Example 1

Preferred Materials

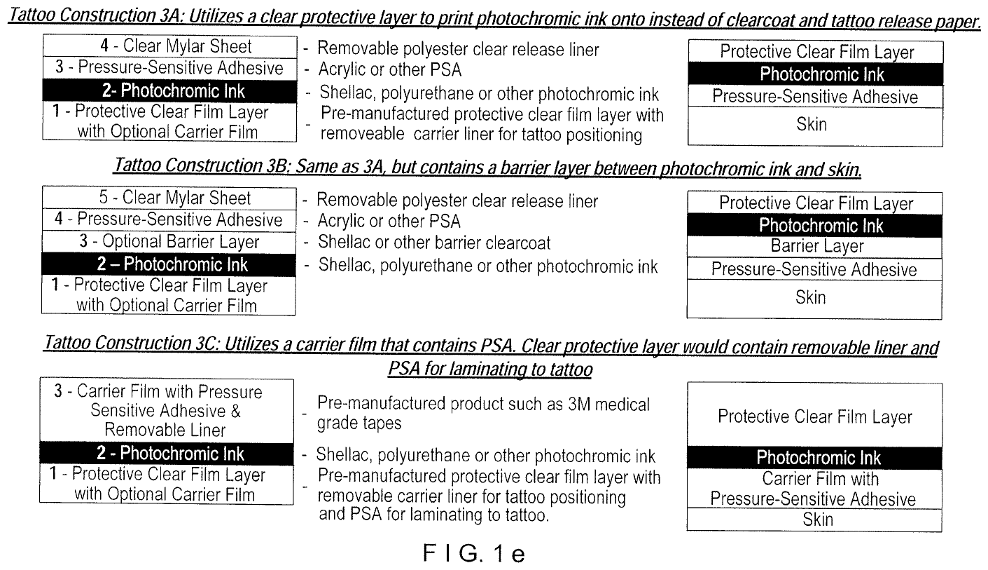

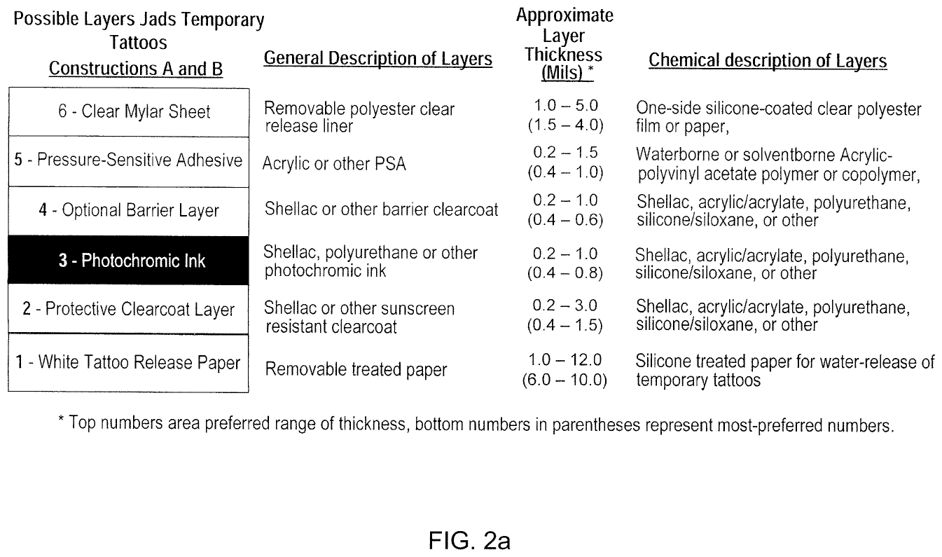

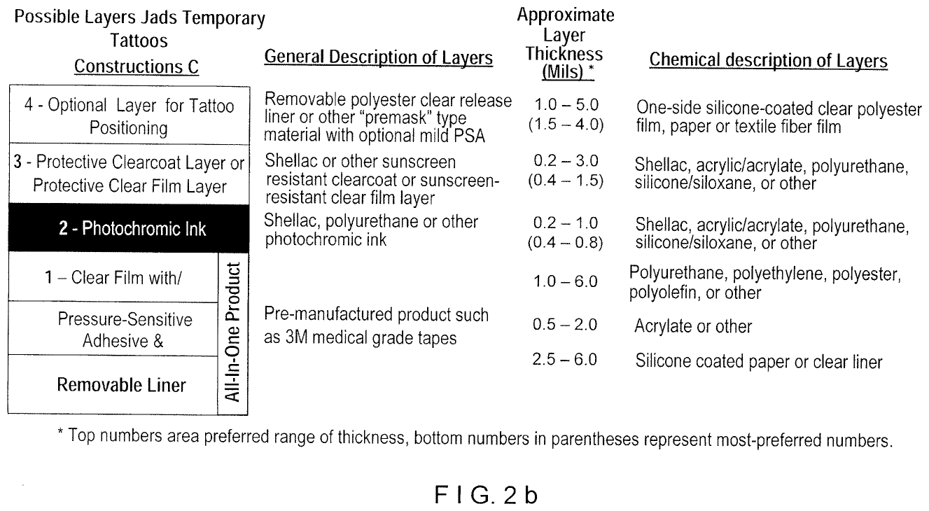

Preferred layers for each of the construction types along with a description of the preferred and most preferred thickness of each of the layers are shown in FIG. 2.

### Example 2

### Test of Effects of Sunscreen Exposure on Tattoo Made with Shellac-Based Photochromic Inks without Clearcoat

Materials:

One yellow tattoo sample was prepared at Stonebridge Coating Laboratories, Inc. of Plymouth, Mich. by drawing down a yellow photochromic ink onto tattoo release paper from The Tattoo Factory. Drawdowns were allowed to dry overnight then an adhesive layer was applied using adhesive sheets in Printable Tattoo Kit made by Silhouette. The sample was cut and identified as 363A—Yellow Triangle Tattoo made with Jads' 2% Photochromic Shellac Ink.

Sunscreens used in the study included Coppertone Waterbabies Lotion SPF 50 and Coppertone Sport Lotion SPF 50.

Sample Preparation:

The tattoo was applied to a thin rubber sheet (synthetic skin for tattoo practice) following manufacturer's recommendations. The clear sheet was removed and tattoo placed upside down onto the rubber sheet. A water saturated paper towel was put over the tattoo paper backside, covered with a 4″×4″ piece of glass and a 1 Kg weight. After 30-35 seconds, the paper was peeled away. The tattoo fully transferred and appeared smooth.

The tattoo was divided into three sections by lines drawn on them with Sharpie marker creating an area to serve as control with no sunscreen and then two other areas where the two sunscreens were applied.

Sunscreens were applied and spread uniformly throughout each section using a gloved finger making circular motions, followed by linear up and down, then side-to-side motions. Gloved fingers were changed to prevent cross-contamination between sunscreens. The sample was immediately photo-activated using an artificial sun lamp, observed for color change, photographed and allowed to set unactivated for one hour. Sample was then washed with hand soap and water, blotted dry, re-activated and photographed.

Test Results:

The tattoo was evaluated for color observations. The yellow ink photo-activated from its colorless appearance to a deep gold/yellow color in the presence of the UV light. The color faded pretty well with the application of sunscreen, but did not turn completely colorless. Upon washing, all color returned on the yellow tattoo.

### Example 3

### Sunscreen Resistance Test with Temporary Tattoos with a Shellac-Based Photochromic Ink

Materials:

One yellow shellac-based ink was tested comprised of shellac, denatured alcohol, photochromic dye predissolved in solvent.

Two sunscreen lotions were used in the study including Coppertone Water Babies Pure & Simple SPF 50 and Coppertone Sport SPF 50.

Sample Preparation:

The ink was drawn down on tattoo release paper using 0.0015″ bird blade applicator. Films were allowed to dry overnight then bonded with a layer of tattoo adhesive applied via squeegee creating a sheet of temporary tattoo material having the following construction:

White Tattoo Release Paper
Photochromic Ink
Pressure-Sensitive Adhesive
Clear Mylar Sheet

Small rectangles were cut from the larger sheets creating the tattoos. They were affixed to the synthetic skin rubber sheets by removing the clear mylar sheet, positioning them onto the substrate, applying a water saturated paper towel folded in 4 with a 4″×4″×¼″ glass plate and 1 kg weight atop for 30 seconds. The tattoo release paper was then removed and the transferred tattoo blotted dry. The resulting tattoo samples were as follows:

Photochromic Ink
Pressure-Sensitive Adhesive
Rubber Sheet or Skin

Three temporary tattoos were affixed to the rubber sheet, top to serve as a control with no sunscreen, middle to be treated with Water babies and bottom sample with Sport 50.

Testing Details:

Sunscreens were applied and spread uniformly throughout each square using a gloved finger making circular motions, followed by linear up and down, then side-to-side motions. Gloved ringers were changed to prevent cross-contamination between sunscreens.

The sunscreens were allowed to remain on the tattoos for 2 hours prior to washing with hand soap, water and clean terry towel. They were photographed before activation, after activation, after sunscreen application and after wash.

Results & Conclusion:

Tattoos transferred well to the rubber sheets resulting in glossy, colorless rectangles. Upon activation with an indoor sunlamp, the tattoos changed from colorless to yellow/gold. The color faded for the most part when sunscreen was applied. After 2 hours, the sunscreens were washed off with soap, water and terry towel then reactivated with the sunlamp. All of the tattoos returned to their original color and appeared to be intact on the rubber sheets.

### Example 4

### Sunscreen Resistance Test with Temporary Tattoos Comprised of Urethane Clearcoats Atop Photochromic Inks

Materials:

Five different urethane-based clearcoats, both glossy and matte finish, were tested atop three different color acrylic/vinyl photochromic inks; blue, violet and yellow.

Two sunscreen lotions were used in the study including Coppertone Water Babies Pure & Simple SPF 50 and Coppertone Sport SPF 50, and one spray sunscreen was used. Coppertone Kids SPF Broad Spectrum 50.

Sample Preparation:

The clearcoats were drawn down on tattoo release paper using 0.0015″ bird blade applicator. Films were allowed to dry for one week then photochromic inks were applied depositing the same 0.0015″ of wet ink onto the dried clearcoat films. Samples were allowed to dry overnight then bonded with a layer of tattoo adhesive applied via squeegee creating a sheet of temporary tattoo material having the following construction:

White Tattoo Release Paper
Protective Clearcoat Layer
Photochromic Ink
Pressure-Sensitive Adhesive
Clear Mylar Sheet

Small rectangles were cut from the larger sheets creating the tattoos. They were affixed to the synthetic skin rubber sheets by removing the clear mylar sheet, positioning them onto the substrate, applying a water saturated paper towel folded in 4 with a 4″×4″×¼″ glass plate and 1 kg weight atop for 30 seconds. The tattoo release paper was then removed and the transferred tattoo blotted dry. The resulting tattoo samples were as follows:

Protective Clearcoat Layer
Photochromic Ink
Pressure-Sensitive Adhesive
Rubber Sheet or Skin

Four sets of fifteen temporary tattoos were affixed to rubber sheets, top row to serve as a control with no sunscreen, middle to be treated with Water babies and bottom sample with Sport 50 and a separate sheet to be sprayed with Coppertone Kids spray sunscreen.

Testing Details:

Sunscreens were applied and spread uniformly throughout each square using a gloved finger making circular motions, followed by linear up and down, then side-to-side motions. Gloved fingers were changed to prevent cross-contamination between sunscreens.

The sunscreens were allowed to remain on the tattoos for 2 hours prior to washing with hand soap, water and clean terry towel. They were photographed before activation, after activation, after sunscreen application and after wash.

Results & Conclusions:

Tattoos transferred well to the rubber sheets resulting in colorless rectangles. Upon activation with an indoor sunlamp, the tattoos changed from colorless to their respective colors; blue, violet and yellow. The color faded somewhat when sunscreen was applied. After 2 hours, the sunscreens were washed off with soap, water and terry towel then reactivated with the sunlamp. All of the tattoos returned to mostly to their original color and appeared to be intact on the rubber sheets, including those with the spray sunscreen.

### Example 5

### Sunscreen Resistance Test with Temporary Tattoos Comprised of Acrylic-Urethane Clearcoat Atop Shellac-Based Photochromic Inks

Materials:

One acrylic-urethane-based clearcoat was tested atop two shellac-based photochromic inks, one yellow and one blue.

Two sunscreen lotions were used in the study including Coppertone Water Babies Pure & Simple SPF 50 and Coppertone Sport SPF 50, and one spray sunscreen was used, Coppertone Kids SPF Broad Spectrum 50.

Sample Preparation:

The clearcoat was drawn down on tattoo release paper using 0.0015″ bird blade applicator. Films were allowed to dry for one week then photochromic inks were applied depositing the same 0.0015″ of wet ink onto the dried clearcoat films. Samples were allowed to dry overnight then bonded with a layer of tattoo adhesive applied via squeegee creating a sheet of temporary tattoo material having the following construction:

White Tattoo Release Paper
Protective Clearcoat Layer
Photochromic Ink
Pressure-Sensitive Adhesive
Clear Mylar Sheet

Small rectangles were cut from the larger sheets creating the tattoos. They were affixed to the synthetic skin rubber sheets by removing the clear mylar sheet, positioning them onto the substrate, applying a water saturated paper towel folded in 4 with a 4″×4″×¼″ glass plate and 1 kg weight atop for 30 seconds. The tattoo release paper was then removed and the transferred tattoo blotted dry. The resulting tattoo samples were as follows:

Protective Clearcoat Layer
Photochromic Ink
Pressure-Sensitive Adhesive
Rubber Sheet or Skin

Four sets of two temporary tattoos were affixed to rubber sheet, top row to serve as a control with no sunscreen, middle to be treated with Water babies and bottom sample with Sport 50 and a separate sheet to be sprayed with Coppertone Kids spray sunscreen.

Testing Details:

Sunscreens were applied and spread uniformly throughout each square using a gloved finger making circular motions, followed by linear up and down, then side-to-side motions. Gloved fingers were changed to prevent cross-contamination between sunscreens.

The sunscreens were allowed to remain on the tattoos for 2 hours prior to washing with hand soap, water and clean terry towel. They were photographed before activation, after activation, after sunscreen application and after wash.

Results & Conclusions:

Tattoos transferred well to the rubber sheets resulting in glossy, colorless rectangles. Upon activation with an indoor sunlamp, the tattoos changed from colorless to their respective colors; blue and yellow. The colors faded to colorless when sunscreen was applied. After 2 hours, the sunscreens were washed off with soap, water and terry towel then reactivated with the sunlamp. All of the tattoos returned to most of their original color and appeared to be intact on the rubber sheets, including those with the spray sunscreen.

Although certain preferred embodiments of the invention have been described herein, it will be apparent to those skilled in the art that variations and modifications may be made without departing from the spirit and scope of the invention as defined by the following claims.

What is claimed is:

1. A temporary tattoo decal comprising:
(a) a porous paper base;
(b) a water soluble slip layer or a release coating on at least a first surface of the paper base;
(c) a photochromic ink layer atop the slip layer or the release coating; and
(d) a pressure-sensitive adhesive layer atop the photochromic ink layer;
wherein the photochromic ink layer comprises at least one photochromic material that is capable of changing its color when exposed to UV radiation and a polymer matrix in which the at least one photochromic material is distributed; and wherein the polymer matrix substantially prevents the ink material from degrading when covered with a sunscreen product and inhibits the photochromic ink from washing off when the sunscreen product covering the photochromic ink layer is washed off; and
wherein the temporary tattoo decal is constructed and arranged such that, when the porous paper base is wetted with water with the pressure-sensitive adhesive layer of the temporary tattoo decal pressed against the skin of a user, the porous paper base and slip layer or release coating can be separated from a remainder of the temporary tattoo decal leaving a photochromic ink image adhered to the skin of the user via the pressure-sensitive adhesive layer.

2. The temporary tattoo decal according to claim 1, further comprising a barrier layer between the photochromic ink layer and the pressure-sensitive adhesive layer that prevents the photochromic material in the photochromic ink layer from possibly migrating to the pressure-sensitive adhesive layer.

3. The temporary tattoo decal according to claim 2, wherein the barrier layer comprises shellac, acrylic, acrylate, polyurethane or cellulose.

4. The temporary tattoo decal according to any one of claims 1-3, further comprising a removable release liner on the pressure-sensitive adhesive layer.

5. The temporary tattoo decal according to any one of claims 1-4, further comprising a transparent sunscreen resistant transparent clear coat layer between the slip layer or release coating and the photochromic ink layer.

6. The temporary tattoo decal according to claim 5, wherein the transparent sunscreen resistant layer comprises a polymer selected from the group consisting of shellac, acrylic, acrylate, polyurethane, silicone and siloxane.

7. The temporary tattoo decal according to any one of claims 1-6, comprising the release coating on the at least first surface of the paper base, the release coating comprising silicone.

8. The temporary tattoo decal according to any one of claims 1-7, wherein the polymer matrix comprises a polymer selected from the group consisting of shellac, polyurethane, acrylic polymer, silicone-containing polymer and a mixture thereof.

9. A temporary tattoo decal comprising:
(f) a porous paper base;
(g) a slip layer or release coating on at least a first surface of the paper base;
(h) a transparent clear coat layer atop the slip layer or release coating;
(i) a photochromic ink layer atop the transparent clear coat layer, wherein the photochromic ink layer comprises (i) at least one photochromic material that is capable of changing its color when exposed to UV radiation and (ii) a polymer matrix in which the at least one photochromic material is distributed; and
(j) a pressure-sensitive adhesive layer covering the photochromic ink layer,
wherein the temporary tattoo decal is constructed and arranged such that, when the porous paper base is wetted with water with the temporary tattoo decal disposed with the pressure-sensitive adhesive layer pressed against the skin of a user, the porous paper base and slip layer or release coating can be separated from a remainder of the temporary tattoo decal leaving a photochromic ink image adhered to the skin of the user via the pressure-sensitive adhesive layer; and
wherein the transparent clear coat layer comprises a material that is constructed and arranged to allow at least a portion of a sunscreen product, when deposited on a top surface of the clear coat layer, to adhere to the clear coat layer while preventing the sunscreen product so deposited from penetrating through the clear coat layer to the photochromic ink layer, the clear coat layer being transparent to allow UV radiation to pass through the clear coat layer to the photochromic ink layer at least when the surface of the clear coat layer is not covered with the sunscreen product.

10. The temporary tattoo decal according to claim 9, further comprising a barrier layer between the photochromic ink layer and the pressure-sensitive adhesive layer that prevents the photochromic material in the photochromic ink layer from migrating to the pressure-sensitive adhesive layer.

11. The temporary tattoo decal according to claim 10, wherein the barrier layer comprises shellac, acrylic, acrylate, polyurethane or cellulose.

12. The temporary tattoo decal according to any one of claims 9-11, further comprising a removable release liner on the pressure-sensitive adhesive layer.

13. The temporary tattoo decal according to claim 12, wherein the removable release liner comprises a silicone-coated polyester film or paper.

14. The temporary tattoo decal according to any one of claims 9-13, wherein the clear coat layer comprises a polymer selected from the group consisting of shellac, acrylic, acrylate, polyurethane, silicone and siloxane.

15. The temporary tattoo decal according to any one of claims 9-14, comprising the release coating on the at least first surface of the paper base, the release coating comprising silicone.

16. The temporary tattoo decal according to any one of claims 9-15, wherein the polymer matrix comprises a polymer selected from the group consisting of shellac, polyurethane, acrylic polymer and a mixture thereof.

17. A temporary tattoo decal comprising:
(a) a carrier film with pressure-sensitive adhesive coating on a first surface thereof;
(b) a photochromic ink layer on a surface of the carrier film that is opposed to the first surface comprising the pressure-sensitive adhesive coating; wherein the photochromic ink layer comprises at least one photochromic material that is capable of changing its color when exposed to UV radiation and a polymer matrix in which the at least one photochromic material is distributed; and wherein the polymer matrix substantially prevents the ink material from degrading when covered with a sunscreen product and inhibits the photochromic ink from washing off when the sunscreen product covering the photochromic ink layer is washed off.

18. The temporary tattoo decal according to claim 17, further comprising a release liner removably attached to the pressure sensitive adhesive coating to protect the pressure sensitive adhesive coating.

19. The temporary tattoo decal according to claim 18, wherein the release liner comprises a silicone-coated polyester film.

20. The temporary tattoo decal according to any one of claims 17-19, further comprising a transparent clear coat layer atop the photochromic ink layer to prevent sunscreen applied to the temporary tattoo decal from penetrating to the photochromic ink layer.

21. The temporary tattoo decal according to claim 20, wherein the clear coat layer comprises a polymer selected from the group consisting of shellac, acrylic, acrylate, polyurethane, silicone and siloxane.

22. A temporary tattoo decal comprising:
(a) a carrier film with pressure-sensitive adhesive coating on a first surface thereof; and
(b) a photochromic ink layer on a surface of the carrier film that is opposed to the first surface comprising the pressure-sensitive adhesive coating; and
(c) a transparent clear coat layer atop the photochromic ink layer;
wherein the photochromic ink layer comprises at least one photochromic material that is capable of changing its color when exposed to UV radiation and a polymer matrix in which the at least one photochromic material is distributed; and
wherein the transparent clear coat layer comprises a material that is constructed and arranged to allow at least a portion of a sunscreen product, when deposited on a top surface of the clear coat layer, to adhere to the clear coat layer while preventing the sunscreen product so deposited from penetrating through the clear coat layer to the photochromic ink layer, the clear coat layer being transparent to allow UV radiation to pass through the clear coat layer to the photochromic ink layer at least when the surface of the clear coat layer is not covered with the sunscreen product.

23. The temporary tattoo decal according to claim 22, further comprising a release liner removably attached to the pressure sensitive adhesive coating to protect the pressure sensitive adhesive coating.

24. The temporary tattoo decal according to claim 23, wherein the release liner comprises a silicone-coated polyester film or paper.

25. The temporary tattoo decal according to any one of claims 22-24, wherein the clear coat layer comprises a polymer selected from the group consisting of shellac, acrylic, acrylate, polyurethane, silicone and siloxane.

26. The temporary tattoo decal according to any one of claims 1-25, wherein the photochromic material comprises a photochromic pigment or dye that is colorless in an inactive state, activates to color when exposed to ultraviolet (UV) light, reverts to colorless when covered with the selected sunscreen product and re-activates to color when the sunscreen product is removed.

27. The temporary tattoo decal according to any one of claims 1-25, wherein the photochromic material comprises a photochromic pigment or dye that starts with an initial color, activates to colorless or to a different color when exposed to UV radiation, reverts to the initial color when covered with sunscreen and re-activates to colorless or to the different color when the selected sunscreen product is removed.

28. The temporary tattoo decal according to any one of claims 1-25, wherein the photochromic material is one in which a color change of the photochromic material is irreversible in that the color of the photochromic material does not reverse after it has been exposed to a predetermined amount of UV radiation.
